(19) United States
(12) Patent Application Publication
(10) Pub. No.: US 2013/0116143 A1
(43) Pub. Date: May 9, 2013

(54) DETECTION AND ANALYSIS OF EPIGENETIC AND GENETIC CHANGES IN TUMOR TISSUE

(71) Applicants: Liying Yan, Ashland, MA (US); Matthew L. Poulin, Framingham, MA (US)

(72) Inventors: Liying Yan, Ashland, MA (US); Matthew L. Poulin, Framingham, MA (US)

(73) Assignee: EPIGENDX, INC., Hopkinton, MA (US)

(21) Appl. No.: 13/640,333

(22) PCT Filed: Apr. 19, 2011
(86) PCT No.: PCT/US11/32973

§ 371 Date: Jan. 18, 2013

Related U.S. Application Data
(60) Provisional application No. 61/325,668, filed on Apr. 19, 2010.
(60) Provisional application No. 61/469,173, filed on Mar. 30, 2011.

(51) Int. Cl. C12Q 1/68

(52) U.S. Cl. CPC C12Q 1/6886
USPC 506/9; 435/6.11; 506/16

(57) ABSTRACT

The invention generally relates to a novel method and related compositions for detecting and analyzing cancer. More particularly, the invention relates to unique methods, compositions and assays useful for diagnosing and measuring the presence and/or risk of ovarian cancer involving the utilization of various generic and epigenetic biomarkers.

### TECHNICAL FIELD OF THE INVENTION

The invention generally relates to a novel method and related compositions for detecting and analyzing cancer. More particularly, the invention relates to unique methods, compositions and assays useful for diagnosing and measuring the presence and/or risk of ovarian cancer.

### BACKGROUND OF THE INVENTION

Ovarian cancer is the fifth leading cause of cancer related deaths and remains the most lethal gynecological cancer. One of the main reasons for such high mortality is the lack of specific screening tests from physical and pelvic exams, and relatively little is known about the molecular events that lead to the development of this highly aggressive disease. The recent discovery of microRNAs (miRNA), a class of small non-coding RNAs that target other mRNAs and triggering translation repression and/or RNA degradation, has revealed the existence of a new level of gene expression regulation. Many studies involving various types of human cancers proved that miRNAs have a definitive role in tumorigenesis.

Various molecular changes have been identified and have shown promise for their diagnostic, prognostic and curative capacity but still need further validation. Vitamin D has been shown to play a role in the suppression of tumor growth and in the modification of some properties of fully transformed malignant cells. En light of evidence for promoter methylation of the vitamin D receptor in the control of the expression of this gene, we have designed methylation assays that cover many regions of this gene in order to determine its methylation profile in normal individuals, ovarian tumors and ovarian cell lines. This gene is highly polymorphic and SNP assays were also designed to analyze the genetic variability within the VDR gene as well. In order to determine if the methylation state and/or the genetic make-up within the VDR gene plays a role in ovarian cancer we analyzed 76 CpG sites and 20 SNPs in normal male blood DNA and tumor sample DNA.

Pyrosequencing is a real-time based sequencing technology that has been used widely for DNA methylation analysis and mutation detection.

### SUMMARY OF THE INVENTION

The invention provides unique methods, compositions and assays useful for diagnosing and measuring the presence and/or risk of ovarian cancer. The invention overcomes the shortcoming of the assays and panels presently available in that the methods disclosed herein enable/allows ovarian cancer screening and early detection.

In one aspect, the invention generally relates to a method for determining the presence or risk of ovarian cancer in a human subject. The method includes: obtaining a test sample from a human subject; analyzing the test sample obtained from the subject for the presence, amount, or both the presence and amount of one or more genetic biomarkers; analyzing the test sample obtained from the subject for the presence, amount, or both the presence and amount of one or more epigenetic biomarkers; and transforming the result of genetic biomarker analysis and the result of epigenetic biomarker analysis into one or more parameters useful in determining the presence or risk of ovarian cancer in the subject.

In some embodiments, at least one of the one or more genetic biomarkers provides gene mutation information of the subject. In some embodiments, the one or more genetic biomarkers are selected from Table 3A and Table 3B and wherein the one or more epigenetic biomarkers are selected from Table 4. In certain preferred embodiments, the one or more genetic biomarkers and one or more epigenetic biomarkers are indicative of a distinctive sub-type of ovarian cancer.

In another aspect, the invention generally relates a multi-marker panel for determining the presence or risk of ovarian cancer in a human subject, the panel comprising one or more genetic biomarkers and one or more epigenetic biomarkers. In some embodiments, the multi-marker panel includes three or more genetic biomarkers and three or more epigenetic biomarkers. In some embodiments, the multi-marker panel includes five or more genetic biomarkers and five or more epigenetic biomarkers.

In yet another aspect, the invention generally relates to a method for determining whether a tumor sample comprises an ovarian cancer cell. The method includes: determining polynucleotide expression levels for one or more of genes selected from Table 5 and determining aberrant methylation levels for one or more of biomarkers from Table 5.

In some embodiments, the method includes: determining polynucleotide expression levels for three or more of genes selected from Table 5 and determining aberrant methylation level for three or more of markers from Table 5. In some embodiments, the method includes: determining polynucleotide expression levels for five or more of genes selected from Table 5 and determining aberrant methylation level for five or more of markers from Table 5.

In yet another aspect, the invention generally relates a method for determining the presence or risk of cancer in a human subject. The method includes: obtaining a test sample from a human subject; analyzing the test sample obtained from the subject for the presence, amount, or both the presence and amount of one or more genetic biomarkers; analyzing the test sample obtained from the subject for the presence, amount, or both the presence and amount of one or more epigenetic biomarkers; and transforming the result of genetic biomarker analysis and the result of epigenetic biomarker analysis into one or more parameters useful in determining the presence or risk of ovarian cancer in the subject.

### BRIEF DESCRIPTION OF THE DRAWINGS

FIG. 1 shows an exemplary global methylation analysis utilizing the human LINE assay.

FIG. 2 shows an exemplary methylation analysis of two miRNA promoters.

FIG. 3 shows an exemplary analysis of human TNFSF7 promoter methylation.

FIG. 4 shows exemplary results from a VDR gene assay.

FIG. 5 shows exemplary results from a VDR gene assay.

FIG. 6 shows exemplary results from a VDR gene assay.

FIG. 7 shows exemplary results of genetic variation analysis.

FIG. 8 shows exemplary results from rs731236 SNP.

FIG. 9A shows exemplary genes with differential expression ovarian vs. normal. FIG. 9B shows exemplary genetic biomarkers with correlation between mutation and methylation in ovarian cancer cells.

FIG. 10 shows exemplary methylation changes and related genes.

FIG. 11 shows exemplary significant genes that show the combination of differential methylation and corresponding differences in gene expression.

FIG. 12 shows exemplary miRNA with differential expression in ovarian vs normal.

FIG. 13 shows exemplary miRNA genes with differential methylation in ovarian vs normal tissue.

### DEFINITIONS

Unless defined otherwise, all technical and scientific terms used herein have the same meaning as commonly understood to one of ordinary skill in the art to which this disclosure belongs.

As used herein and in the appended claims, the singular forms “a,” “and,” and “the” include plural referents unless the context clearly dictates otherwise. Thus, for example, reference to “a polynucleotide” includes a plurality of such polynucleotides and reference to “the SNP” includes reference to one or more SNPs known to those skilled in the art, and so forth.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

The term “biomarker”, as used herein, refers to anatomic, physiologic, biochemical, or molecular parameters associated with the presence and severity of specific disease states. Broadly defined, a biomarker is a biological indicator that may be deliberately used by an observer or instrument to reveal, detect, or measure the presence or frequency and/or amount of a specific condition, event or substance. For example, a specific and unique sequence of nucleotide bases may be used as a genetic marker to track patterns of genetic inheritance among individuals and through families. Similarly, molecular markers are specific molecules, such as proteins or protein fragments, whose presence within a cell or tissue indicates a particular disease state. For example, proliferating cancer cells may express novel cell-surface proteins not found on normal cells of the same type, or may over-express specific secretory proteins whose increased or decreased abundance (e.g., over expression or under expression, respectively) can serve as markers for a particular disease state. Biomarkers includes cancer biomarkers (i.e. PSA, etc.), cardiovascular disease biomarkers (i.e. troponin, CKMB, myoglobin, etc.), therapeutic drug monitoring biomarkers, etc.

The terms “complementary”, are used herein, refer to the sequences of polynucleotides which are capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The terms “epigenetic modification” or “epigenetic change”, as used herein, refer to an inheritable or non-inheritable change in gene function that occurs without a change in the DNA sequence. Epigenetic modifications include DNA methylation, histone modification (e.g., acetylation), and small RNA interference, etc. In addition, “epigenetic modification” or “epigenetic change” as used herein may also include chromosomal binding of proteins that are responsible for DNA methylation, histone modification (e.g., acetylation), and small RNA, such as miRNA, interference, etc., as well as proteins that binds to modified histones or methylated DNA. Frequently, the epigenetic change will result in an alteration in the levels of expression of the gene which may be detected (at the RNA or protein level as appropriate) as an indication of the epigenetic change. Often the epigenetic change results in silencing or down regulation of the gene, referred to herein as “epigenetic silencing”. The most frequently investigated epigenetic change in the methods of the invention involves determining the methylation status of the gene, where an increased level of methylation is typically associated with the relevant cancer (since it may cause down regulation of gene expression).

The term “gene”, as used herein, refers to a segment of genomic DNA that contains the coding sequence for a protein, wherein the segment may include promoters, exons, introns, and other untranslated regions that control expression.

The term “genotype”, as used herein, refers to an unphased 5′ to 3′ sequence of . nucleotide pair(s) found at a set of one or more polymorphic sites in a locus on a pair of homologous chromosomes in a subject.

The term “genotyping” a sample or a subject for a polymorphism, as used herein, involves determining the specific allele or the specific nucleotide(s) carried by an individual at a biallelic marker.

The term “isolated”, as used herein, requires that the material be removed from its original environment (e.g., the natural environment if it is naturally occurring). For example, naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The terms “level of expression” or “expression level”, as used herein, refer to the amount of a polynucleotide or an amino acid product or protein in a biological sample. “Expression” generally refers to the process by which gene-encoded information is converted into the structures present and operating in the cell. Therefore, according to the invention “expression” of a gene may refer to transcription into a polynucleotide, translation into a protein, or even post-translational modification of the protein. Fragments of the transcribed polynucleotide, the translated protein, or the post-translationally modified protein shall also be regarded as expressed, whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

The terms “locus” or “loci”, as used herein, refer to a region or regions, respectively, of genomic DNA with definable attributes, such as being associated with a particular phenotype by genetic mapping techniques. For example, human alpha satellite DNAs are considered to be centromeric loci. The term “imprinted locus” is used to indicate a region of genomic DNA which has expression characteristics that differ from the corresponding homologus allele based on the parental origin of each allele. For example, imprinted loci sometimes differ in gene expression due to differences in DNA methylation or histone acetylation states in their promoter and/or enhancer regions.

The term “mutation”, as used herein, refers to a difference in DNA sequence between or among different genomes or individuals that causes a functional change and which can have a frequency below 1%. Sequence variants describe any alteration in DNA sequence regardless of function or frequency.

The term “nucleotide”, as used herein as an adjective to describe molecules, refers to RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide.

The terms “oligonucleotides” and “polynucleotides”, as used herein, include RNA, DNA, or RNA/DNA hybrid sequences of more than one nucleotide in either single chain or duplex form.

The terms “peptide”, “protein”, “polypeptide”, as used herein, refer to a series of amino acid residues connected to the other by peptide bonds between the alpha-amino and carboxy groups of adjacent residues.

The term “phenotype”, as used herein, refers to any biochemically, anatomically, and clinically distinguishable, detectable or otherwise measurable property of an organism such as symptoms of, or susceptibility to a disease for example. Typically, the term “phenotype” is used herein to refer to symptoms of, or susceptibility to a cardiovascular disorder; or to refer to an individual's response to a therapeutic agent; or to refer to symptoms of, or susceptibility to side effects to a therapeutic agent. A “less severe phenotype” is defined as a less severe form of a cardiovascular disorder, or a form of the cardiovascular disorder that is more responsive to treatment, displays less side effects with treatment, has better prognosis, is not recurrent, or has a combination of these characteristics. A “more severe phenotype” is defined as a more severe form of a cardiovascular disorder, or a form of the disorder that is less responsive to treatment, displays more side effects with treatment, has worse prognosis, is recurrent, or has a combination of these characteristics. In general, the more severe phenotype is a disease state with profound consequences to the patient's life quality and requires more aggressive therapy.

The term “polymorphism”, as used herein, refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A polymorphism may comprise a substitution, deletion or insertion of one or more nucleotides. A single nucleotide polymorphism (SNP) is a single base pair change. Typically, a single nucleotide polymorphism is the replacement of one nucleotide by another nucleotide at the polymorphic site. Deletion of a single nucleotide or insertion of a single nucleotide, also give rise to single nucleotide polymorphisms. In the context of the present disclosure, “single nucleotide polymorphism” refers to a single nucleotide substitution. Typically, between different genomes or between different individuals, the polymorphic site may be occupied by two different nucleotides.

The term “primer”, as used herein, refers to a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase, or in a single nucleotide extension reaction for the measurement of AEI.

The term “purified”, as used herein, refers to a polynucleotide or polynucleotide vector of the disclosure which has been separated from other compounds including, but not limited to other nucleic acids, carbohydrates, lipids and proteins (such as the enzymes used in the synthesis of the polynucleotide), or the separation of covalently closed polynucleotides from linear polynucleotides.

### DETAILED DESCRIPTION OF THE INVENTION

The invention provides unique methods, compositions and assays useful for diagnosing and measuring the presence and/or risk of ovarian cancer. The invention overcomes the shortcoming of the assays and panels presently available in that the methods disclosed herein enable/allows ovarian cancer screening and early detection.

In various embodiments of the invention disclosed herein, a method is provided wherein biomarkers are used to assess the initiation, progression or severity of disease. In general a biomarker can be any biological feature or variable whose qualitative or quantitative presence, absence, or level in a biological system such as a human is an indicator of a biological state of the system. For example, a biomarker of an organism can be useful alone or in combination with other biomarkers and/or clinical factors, to measure the initiation, progression, severity, pathology, aggressiveness, grade, activity, disease sub-classification or other underlying features of one or more biological processes, pathogenic processed, diseased, or responses to therapeutic intervention. Accordingly, biomarkers can be useful to assess the health state or status of an individual by comparing the measured level of one or more biomarkers in a patient or a patient sample to a control. In addition, multiple biomarker levels can be analyzed using a weighted analysis or algorithm to generate a “score” for an individual. The score can be indicative of the disease state of the individual.

Any biological compound that is present in a sample and that can be isolated from or measured in the sample can be potentially used as a biomarker. For examples, SNPs, differential gene expression, differential miRNA expression, and differential methylation of genes and miRNA can be used as biomarker(s).

The level or amount of a biomarker can be determined by any method known in the art and will depend in part on the nature of the biomarker. It should be understood that the amount of the biomarker need not be determined in absolute terms, but can be determined in relative terms.

The development and maintenance of an organism is orchestrated by a set of biochemical reactions and processes that switch parts of the genome off and on at strategic times and locations. Epigenetics is the study of these reactions and the factors that influence the phenotype (appearance) or gene expression caused by mechanisms other than changes in the underlying DNA sequence. These changes may remain through cell divisions for the remainder of the cell's life and may also last for multiple generations. However, there is no change in the underlying DNA sequence of the organism; instead, non-genetic factors cause the organism's genes to behave (or “express themselves”) differently.

The molecular basis of epigenetics involves modifications of the activation of certain genes, but not the basic structure of DNA. The chromatin proteins associated with DNA may be activated or silenced. Epigenetic changes are preserved when cells divide. Specific epigenetic processes include paramutation, bookmarking, imprinting, gene silencing, X chromosome inactivation, position effect, reprogramming, transfection, maternal effects, the progress of carcinogenesis, many effects of teratogens, regulation of histone modifications and heterochromatin, and technical limitations affecting parthenogenesis and cloning.

Different types of epigenetic modifications are closely linked and often act in self-reinforcing manner in the regulation of different cellular processes. There are several layers of regulation of gene expression. One way that genes are regulated is through the remodeling of chromatin. If the way that DNA is wrapped around the histones changes, gene expression can change as well. Chromatin remodeling is accomplished through two main mechanisms. The first way is post translational modification of the amino acids that make up histone proteins. Histone proteins are made up of long chains of amino acids. If the amino acids of histone proteins are changed, the shape of the histone sphere might be modified. DNA is not completely unwound during replication.

The second way is the addition or removal of methyl groups to or from the DNA, mostly at CpG sites, to convert cytosine to 5-methylcytosine. 5-Methylcytosine performs much like a regular cytosine, pairing up with a guanine. However, some areas of genome are methylated more heavily than others and highly methylated areas tend to be less transcriptionally active.

DNA methylation and histone acetylation are major epigenetic modifications that are dynamically linked in the epigenetic control of gene expression and their deregulation plays an important role in tumorigenesis. (Feinberg, et al. 2006 Nat. Rev. Genet. 7:21-33; Jones et al. 2002 Nat. Rev. Genet. 3:415-428.)

DNA methylation is an important regulator of gene transcription and a large body of evidence has demonstrated that aberrant DNA methylation is associated with unscheduled gene silencing, and the genes with high levels of 5-methylcytosine in their promoter region are transcriptionally silent. DNA methylation is essential during embryonic development, and in somatic cells, patterns of DNA methylation are generally transmitted to daughter cells with a high fidelity. Aberrant DNA methylation patterns have been associated with a large number of human malignancies and found in two distinct forms: hypermethylation and hypomethylation compared to normal tissue. Hypermethylation is one of the major epigenetic modifications that repress transcription via promoter region of tumour suppressor genes. Hypermethylation typically occurs at CpG islands in the promoter region and is associated with gene inactivation. Global hypomethylation has also been implicated in the development and progression of cancer through different mechanisms.

MicroRNAs (miRNAs) are short ribonucleic acid (RNA) molecules, on average only 22 nucleotides long. miRNAs are post-transcriptional regulator's that bind to complementary sequences on target messenger RNA transcripts (mRNAs), usually resulting in translational repression and gene silencing. miRNAs) can contribute to cancer development and progression by acting as oncogenes or tumor suppressor genes. Recent studies have also linked different sets of miRNAs to metastasis through either the promotion or suppression of this malignant process. Epigenetic silencing of miRNAs with tumor suppressor features by CpG island hypermethylation is also emerging as a common hallmark of human tumors.

Gene-expression profiling with the use of DNA microarrays allows measurement of messenger RNA (mRNA) transcripts. Results of such studies, for example, have confirmed that breast cancer is not a single disease with variable morphologic features and biomarkers, rather, it is a group of molecularly distinct neoplastic disorders. Profiling results also support the hypothesis that estrogen-receptor (ER)-negative and ER-positive breast cancers originate from distinct cell types and point to biologic processes that govern metastatic progression.

Pyrosequencing is a sequencing-by-synthesis method producing an enzymatic cascade which generated light which is detected as signal. Briefly, pyrophosphate released upon the addition of a nucleotide base during primer extension is converted to ATP. The generated ATP drives the luciferase mediated conversion of luciferin to oxyluciferin generating visible light in amount that is proportional to the amount of ATP.

Early detection of a specific disease state and early treatment can greatly improve a patient's chance for survival while the disease is still localized and its pathologic effects limited anatomically and physiologically.

In the case of cancer, it is a neoplastic disease where cancer cells, unlike benign tumor cells, exhibit the properties of invasion and metastasis and are highly anaplastic. Cancer is characterized by uncontrolled cell proliferation and other malignant cellular properties. Cancer cells can arise from a number of genetic and epigenetic perturbations that cause defects in mechanisms controlling cell migration, invasion, proliferation, survival, differentiation, and growth that lead to tumor formation and/or metastasis. As used herein, the term cancer includes, but is not limited to, the following types of cancer: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art. In one embodiment the cancer is melanoma. In one embodiment the cancer is prostate cancer. In one embodiment the cancer is lung cancer. In one embodiment the cancer is breast cancer.

In one aspect, the invention generally relates to a method for determining the presence or risk of ovarian cancer in a human subject. The method includes: obtaining a test sample from a human subject; analyzing the test sample obtained from the subject for the presence, amount, or both the presence and amount of one or more genetic biomarkers; analyzing the test sample obtained from the subject for the presence, amount, or both the presence and amount of one or more epigenetic biomarkers; and transforming the result of genetic biomarker analysis and the result of epigenetic biomarker analysis into one or more parameters useful in determining the presence or risk of ovarian cancer in the subject.

In some embodiments, at least one of the one or more genetic biomarkers provides gene mutation information of the subject.

In some embodiments, at least one of the one or more epigenetic biomarkers provides miRNA expression information of the subject.

In some embodiments, at least one of the one or more epigenetic biomarkers provides aberrant DNA methylation information of the subject.

In some embodiments, the one or more genetic biomarkers are selected from Table 3A and Table 3B and wherein the one or more epigenetic biomarkers are selected from Table 4.

In certain preferred embodiments, three or more genetic biomarkers selected from Table 3A and Table 3B and three or more epigenetic biomarkers selected from Table 4.

In certain preferred embodiments, five or more genetic biomarkers selected from Table 3A and Table 3B and five or more epigenetic biomarkers selected from Table 4.

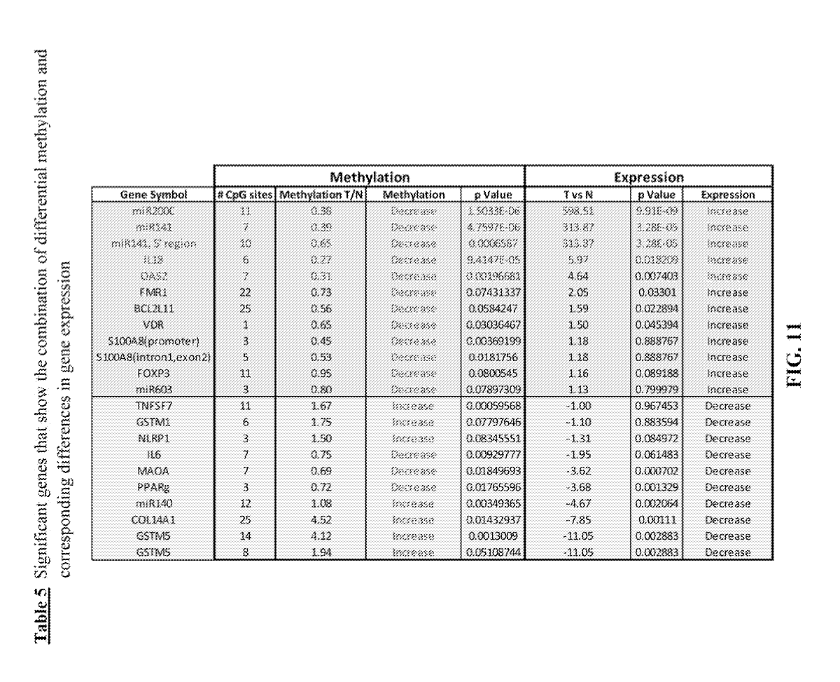

In certain preferred embodiments, genetic biomarkers and epigenetic biomarkers are selected from Table 5 (FIG. 11).

In certain preferred embodiments, the one or more genetic biomarkers and one or more epigenetic biomarkers are indicative of a distinctive sub-type of ovarian cancer including: surface epithelial-stromal tumors, sex cord-gonadal stromal tumors, and germ cell tumors.

In another aspect, the invention generally relates a multi-marker panel for determining the presence or risk of ovarian cancer in a human subject, the panel comprising one or more genetic biomarkers and one or more epigenetic biomarkers.

In some embodiments, the multi-marker panel includes three or more genetic biomarkers and three or more epigenetic biomarkers.

In some embodiments, the multi-marker panel includes five or more genetic biomarkers and five or more epigenetic biomarkers.

In certain preferred embodiments of the invention, the at least one of the one or more genetic biomarkers provides gene mutation information of the subject.

In certain preferred embodiments of the invention, the at least one of the one or more epigenetic biomarkers provides miRNA expression information of the subject.

In certain preferred embodiments, the at least one of the one or more epigenetic biomarker provides aberrant DNA methylation information of the subject.

In certain preferred embodiments, the one or more genetic biomarkers are selected from Table 3A and Table 3B and wherein the one or more epigenetic biomarkers are selected from Table 4.

In yet another aspect, the invention generally relates to a method for determining whether a tumor sample comprises an ovarian cancer cell. The method includes: determining polynucleotide expression levels for one or more of genes selected from Table 5 and determining aberrant methylation levels for one or more of biomarkers from Table 5.

In some embodiments, the method includes: determining polynucleotide expression levels for three or more of genes selected from Table 5 and determining aberrant methylation level for three or more of markers from Table 5.

In some embodiments, the method includes: determining polynucleotide expression levels for five or more of genes selected from Table 5 and determining aberrant methylation level for five or more of markers from Table 5.

In yet another aspect, the invention generally relates a method for determining the presence or risk of cancer in a human subject. The method includes: obtaining a test sample from a human subject; analyzing the test sample obtained from the subject for the presence, amount, or both the presence and amount of one or more genetic biomarkers; analyzing the test sample obtained from the subject for the presence, amount, or both the presence and amount of one or more epigenetic biomarkers; and transforming the result of genetic biomarker analysis and the result of epigenetic biomarker analysis into one or more parameters useful in determining the presence or risk of ovarian cancer in the subject.

In certain preferred embodiments of the invention, the cancer is ovarian cancer, breast cancer, colon cancer and cervical cancer.

### EXAMPLES

### Example 1

Methods disclosed here include DNA gene promoter methylation assays using Pyrosequencing technology, which for example have used to quantify the methylation states of over 20 genes in ten ovarian tumor tissues and their corresponding normal tissue. These candidate genes are known to be related to tumori-genesis. A LINE element repeat assay was also used to analyze global methylation. Additionally, we developed assays in the regions surrounding several miRNAs that have been shown to have altered regulation in ovarian tumors.

Several miRNA promoters were analyzed for methlyation in ovarian tumor and normal tissue. Two miRNA promoters were shown to hypomethylate in tumor tissue when compared with their corresponding normal tissue. This is also the case when the methylation of global methylation marker, LINE promoter, is analyzed. The human TNFSF7 promoter shows hypermethylation in the tumor tissue as compared to the normal tissue. Genetic variations at several loci that are often relevant in the generation of cancerous tissue were also examined. The KRAS, BRAF, and NRAS assays showed no mutations in the ovarian cell lines or tissue; however, there was much variation in members of the cytochrome gene family.

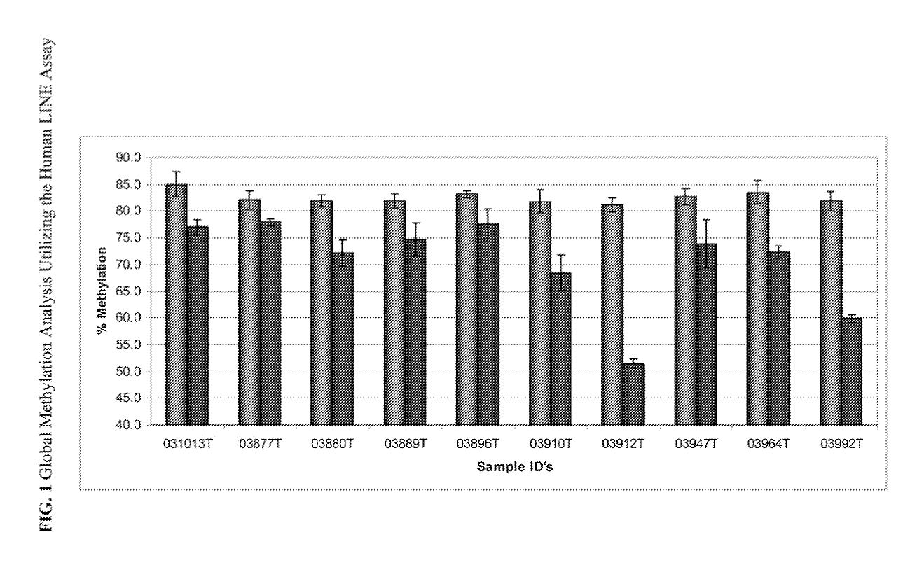

A global methylation analysis utilizing the Human LINE AssayHuman LINE is shown in FIG. 1. Ten ovarian tumor DNA samples (red bars) and their corresponding normal DNA (blue bars) were bisulfite treated and analyzed utilizing the human LINE pyrosequencing assay. The results of triplicate samples were given as the average percent methylation of four CpG sites.

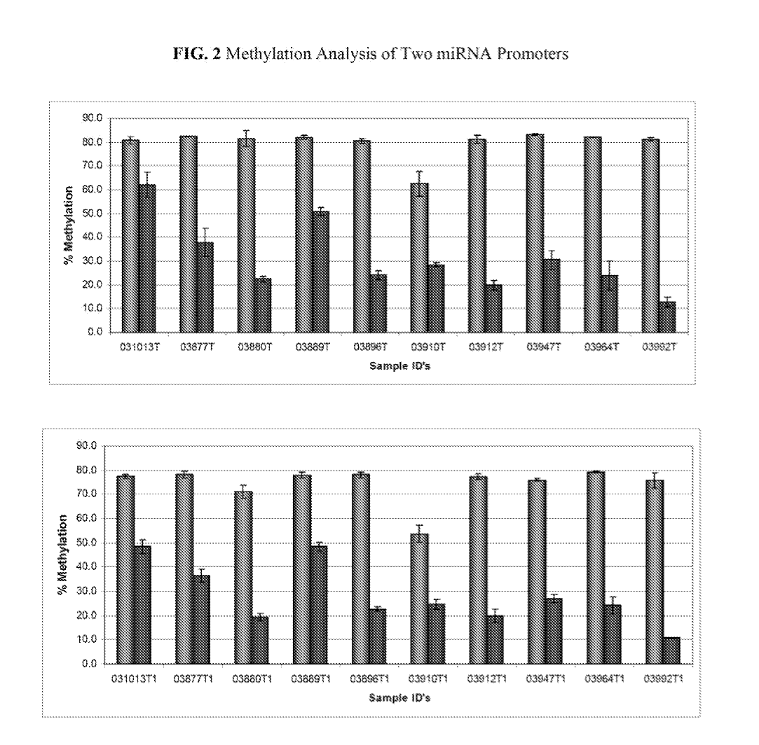

FIG. 2 shows the results from a methylation analysis (percent methylation) of two miRNA promoters. Pyrosequencing results for two miRNA promoters is shown for ten ovarian tumors (red bars) and their corresponding normal tissue (blue bars). Both miRNA promoters have very high methylation in normal ovarian tissue, usually resulting in low levels of expression. The tumor tissues show lower, more variable methylation, which may result in the expression off of these promoters when normally they would be silent.

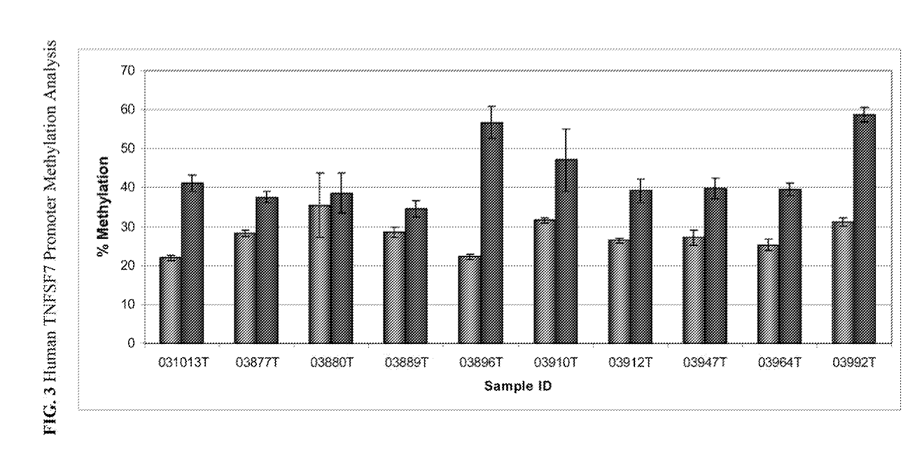

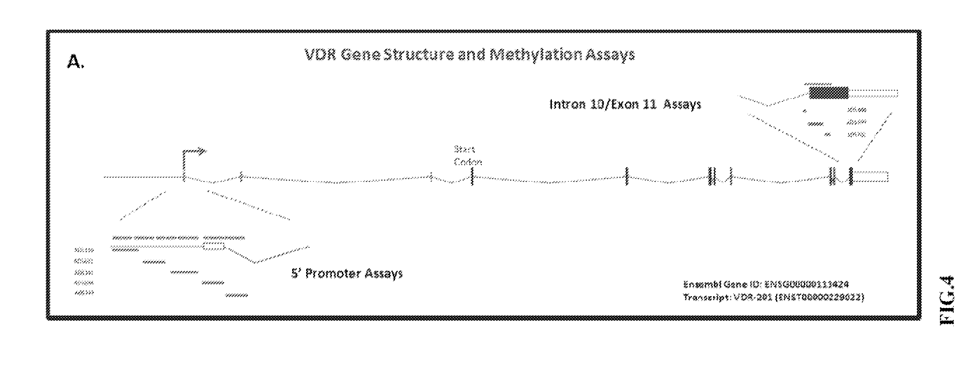

FIG. 3 shows the results from a human TNFSF7 promoter methylation analysis. Ten ovarian tumor DNA samples (red bars) and their corresponding normal DNA (blue bars) were bisulfite treated and analyzed utilizing the human TNFSF7 promoter pyrosequencing assay. The samples were run in triplicate and the data shows the average percent methylation across 11 CpG sites within the promoter. Ovarian tumor DNA consistently showed hypermethylation of this promoter when compared with normal ovarian tissue DNA.

Table 1 (FIG. 7) shows the results from a genetic variation analysis. Single nucleotide polymorphisms were analyzed in eight genes for both ovarian tumor and normal tissue as well as seven ovarian cell lines by the Pyrosequencing PSQ 96 System.

These results demonstrate that Pyrosequencing can be used to detect the hypo and hypermethylation of specific promoters in tumor and non tumorous, normal tissue. The ovarian tumor DNA was shown to be globally hypomethylated by analysis with the LINE promoter assay. Two specific miRNA promoters were shown to be hypomethylated in the tumor tissue as well indicating the possibility that the tumor tissue may be activating the expression of these RNAs. In contrast, the TNFSF7 promoter was hypermethylated in tumor tissue indicating the possibility of a silencing of expression of this gene.

### Example 2

Methylation assay was developed for the VDR promoter and exon 11 region. DNA was purified from 10 ovarian tumor tissue samples and their corresponding normal tissue. Cell line DNA was commercially purchased. 500 ng of DNA was bisulfite treated and purified prior to PCR amplification. Pyrosequencing® was carried out on a PSQ HS 96 pyrosequencing machine and analyzed using Pyro-Q-CpG software. Results are expressed as percent methylation over the region of an assay.

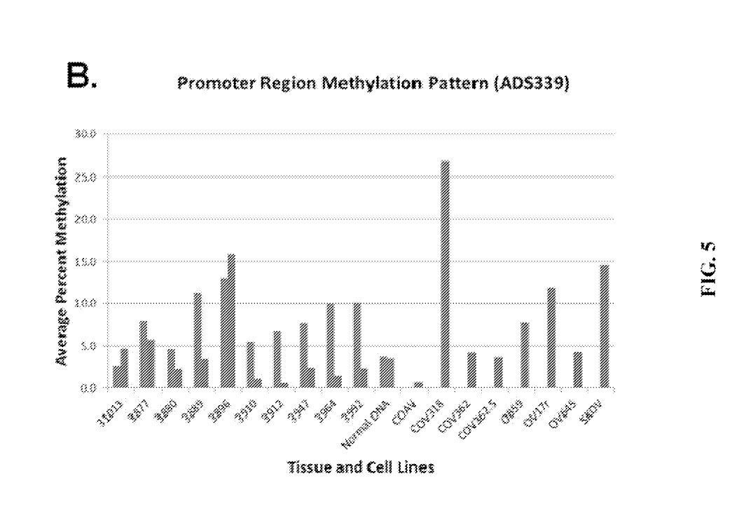

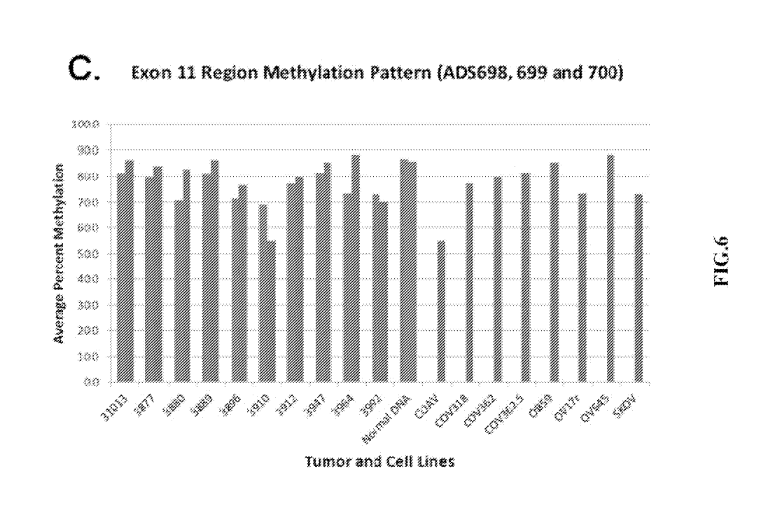

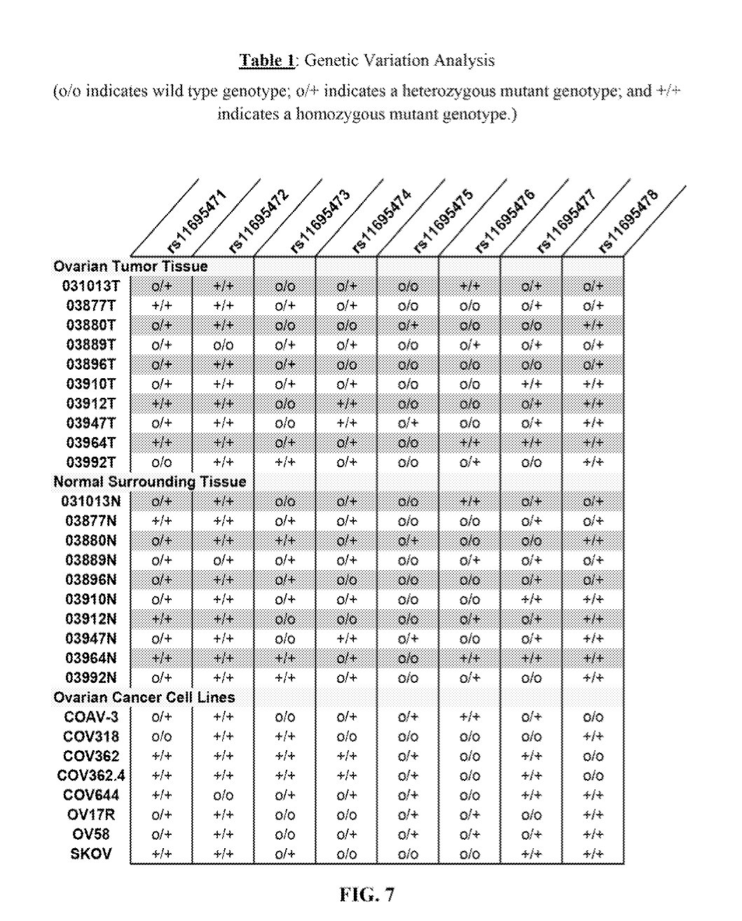

FIGS. 4-6 show certain VDR gene assays and results. In FIG. 4, the VDR gene structure and methylation assays are shown. The transcriptional start site is indicated by the arrow in exon 1 and the translational start codon is in exon 4. Non-coding exons are shown as hollow rectangular boxes while coding exons are solid rectangles and introns are represented by thin lines. Green bars represent areas covered by our methylation assays. In FIG. 5 and FIG. 6, the average percent methylation is shown for normal (blue bars) vs tumor DNA samples as well as ovarian cell lines (red bars). FIG. 5 dipicts the methylation of the promoter region from −723 to −545 of the transcriptional start site and covers 18 CpG sites. FIG. 6 shows the methylation of the intron 10/exon 11 boundary by the assays shown in FIG. 4 and covers 20 CpG sites. Affymetrix array data indicates that there is a 1.5 fold increase in expression (p=0.045) of the VDR gene in tumor tissue compared to normal ovarian tissue.

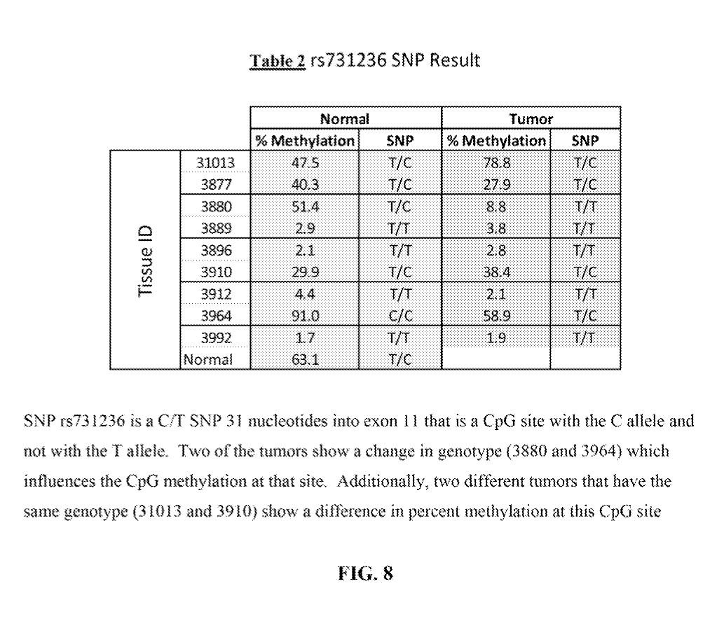

SNP rs731236 is a C/T SNP 31 nucleotides into exon 11 that is a CpG site with the C allele and not with the T allele. Two of the tumors show a change in genotype (3880 and 3964) which influences the CpG methylation at that site. Additionally, two different tumors that have the same genotype (31013 and 3910) show a difference in percent methylation at this CpG site. Results are shown in Table 2 (FIG. 8).

The Vitamin D receptor may have many levels of gene regulation. In a specific promoter region of the VDR receptor there is a decrease in methylation of ovarian tumor DNA compared to normal tissue. Expression data indicates that there is an increase in VDR expression in tumors as well suggesting a possible connection between promoter methylation and gene expression. This is in direct contrast with reported hypermethylation and down regulation of VDR in breast cancer cell lines and breast tumor tissue.

Cell line methylation varied greatly. The 3′ end of the gene at exon 11 had very high levels of methylation and showed a modest increase in methylation of tumor DNA when compared with corresponding normal tissue DNA. There was a C/T SNP in this exon 11 region that creates or destroys a CpG site that may be differentially methylated in ovarian tumor tissue compared with normal ovarian tissue.

### Example 3

Single nucleotide polymorphisms associated with ovarian cancer (cancer tissue compared to control tissues) was studied. A purified nucleic acid fraction of a sample (e.g., frozen tumor biopsy, frozen biopsy of surrounding normal tissue, cancer cell culture, circulating PMLS) was prepared using standard commercially available column isolation methods. DNA isolated from cancer tissue and surrounding normal tissue was subjected to genotyping analysis for the detection of single nucleotide polymorphisms (SNPS). Many methods for detecting SNPs are well known and can be used with the present teachings. Examples of such assays include genotyping microarray analysis, sequencing analysis, including short read sequencing analysis using Pyrosequncingand polymerase chain reaction followed by high resolution melt analysis (HRM) and TaqMan analysis.

Genotyping of the purified DNA was accomplished by hybridization to an Affymatrix SNP array. The SNP array contains short nucleic acid probes for genotyping across 906,600 SNPs. Table 3B (FIG. 3B) shows certain genetic biomarkers with correlation between mutation and methylation in ovarian cancer cells.

### Example 4

Differential methylation of gene set in cancer tissues was compared to control tissues. A purified nucleic acid fraction of a sample (e.g. frozen tumor biopsy, frozen biopsy of surrounding normal tissue, cancer cell culture, circulating PMLS) were subject to bisulfite modification to enable detection and analysis of methylated based. Bisulfite treatment of the DNA and subsequent purification of the modified DNA are conducted using standard commercially available kit products. Many methods for measuring DNA methylation are well known and can be used with the present teaching. All methods require an initial bisulfite modification of the nucleic acid which results in unmethylated cytosine being converted to uracil. This conversion in essence creates a polymorphism which is subsequently measured and representative of the methylation level in the starting DNA. Examples of such methods include bisulfite sequencing, including Pyrosequencing, methylation specific PCR and high resolution melt analysis of PCR amplified bisulfite DNA.

In this example, bisulfite modified DNA from tumor or normal tissue was subjected to methylation analysis using Pyrosequencing. Gene specific primers were used to amplify gene specific regions using polymerase chain reaction (PCR). The amplified products were subjected to quantitiative sequencing using Pyrosequencing technology.

The methylation at each CpG site contained within the gene region was quantified as a percentage of the amount of unmethylated cytosine at each site in the starting material. The percentage of methylation at each CpG site in the gene region was averaged together to provide an average methylation level for each gene.

The results are as follows in Table 4. Genes whose methylation is associated with ovarian cancer specifically. The FMR 1 gene displayed hypomethylation in ovarian tumor DNA only and not in the other tumor types. Genes whose methylation is associated with cancer (common to ovarian, breast and colonrectal) vs normal. The genes in table three showed differential methylation between tumor and corresponding normal DNA. Some were consistentlyhyper or hypomethlated in the four tumor types we examined compared with normal tissue (All Tumor) while others showed some tumor types hypomethylating and others hypermethylatingin the four tumors (Variable).

### Example 5

Many methods for detecting expression levels of a gene transcript (e.g., mRNA, miRNA), with or without quantification, are well known and can be used with the present teachings. One such method is hybridization to nucleic assay probe arrays. A purified ribonucleic acid fraction of a sample (e.g. frozen tumor biopsy, frozen biopsy of surrounding normal tissue, cancer cell culture, circulating PMLS) was prepared using standard commercially available column isolation methods. The purified RNA fraction contained both messenger RNA (mRNA) and other small noncoding RNA transcripts. RNA isolated from cancer tissue and/or surrounding normal tissue was subjected to mRNA expression analysis via hybridization to the Affymatrix Human Exon 1 ST array containing nucleic acid probes for 28, 869 expressed genes. A list of genes with differential mRNA expression in cancer vs normal tissue was obtained.

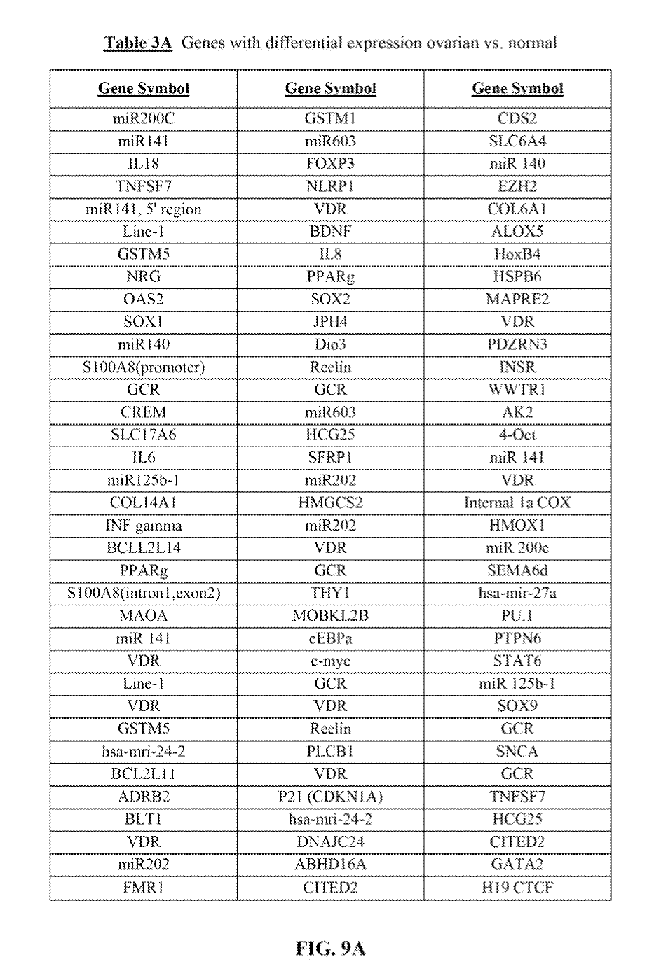

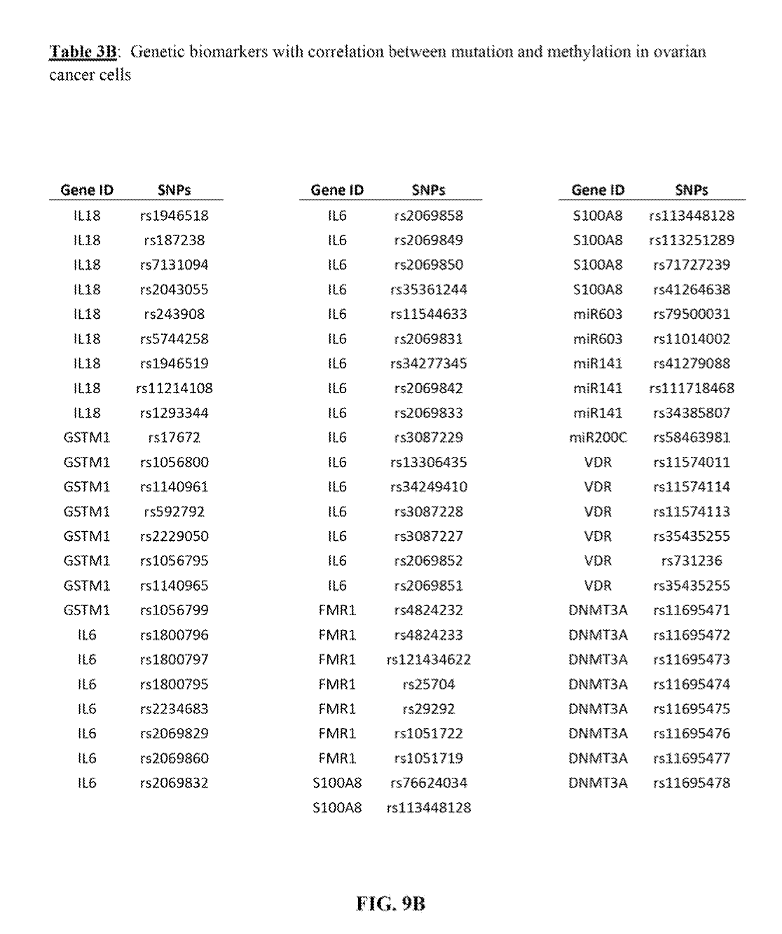

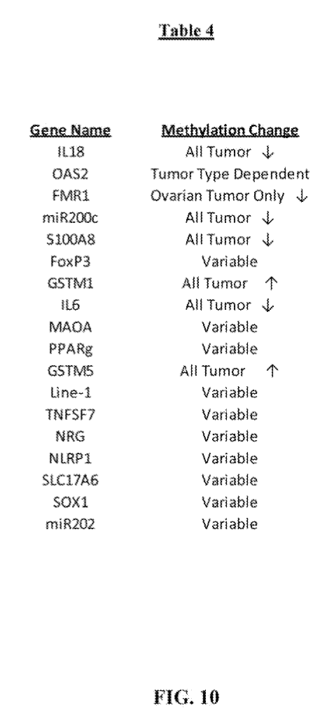

Detection of the hybridization to the array chip is achieved using the TheGeneChip® Laser Scanner 3000 7G. Analysis of the array data is performed with software designed to interpret micro array hybridization data using various algorithms. Results are shown in the Table 3A (FIG. 9A).

### Example 4

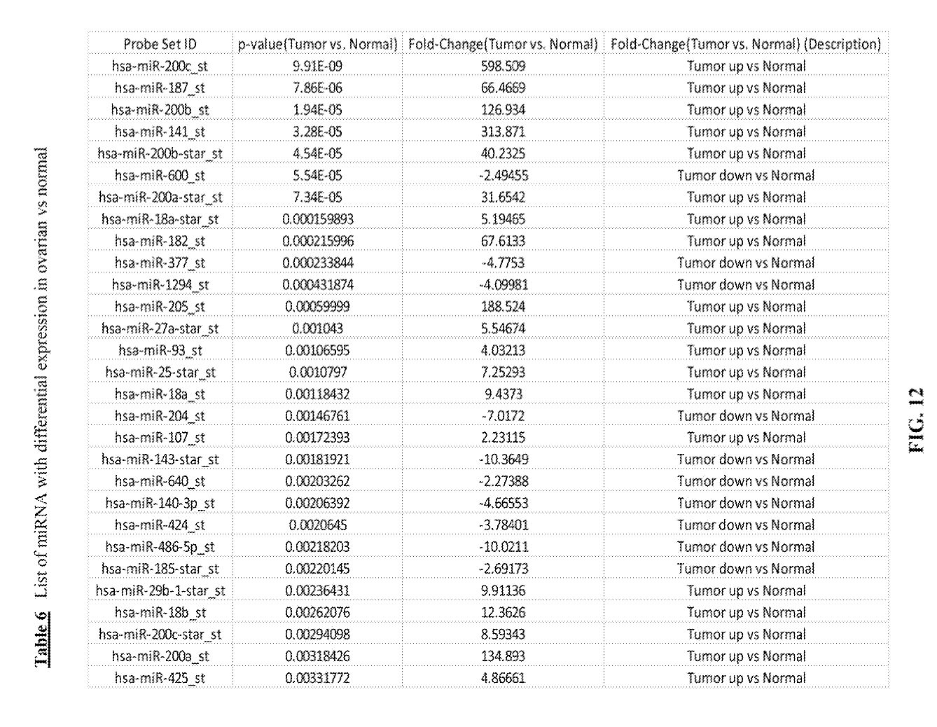

A list of miRNA genes with differential expression in ovarian cancer tissues was obtained. A purified ribonucleic acid fraction of a sample (e.g. frozen tumor biopsy, frozen biopsy of surrounding normal tissue, cancer cell culture, circulating PMLS) was prepared using standard commercially available column isolation methods. The purified RNA fraction contained both messenger RNA (mRNA) and other small noncoding RNA transcripts. RNA isolated from cancer tissue and/or surrounding normal tissue was subjected to expression analysis via hybridization to the Affymatrix GeneChipmiRNA 2.0 array containing 1500 probe sets representing 100% coverage of themiRBase V15, a searchable database of published miRNA sequences and annotation developed at the University of Manchester, Manchester England. The results are shown in the Table 6 (FIG. 12).

### Example 5

A list of miRNA genes with differential methylation in ovarian cancer tissues was obtained. A purified nucleic acid fraction of a sample (e.g. frozen tumor biopsy, frozen biopsy of surrounding normal tissue, cancer cell culture, circulating PMLS) were subject to bisulfite modification to enable detection and analysis of methylated based. Bisulfite treatment of the DNA and subsequent purification of the modified DNA were conducted using standard commercially available kit products. Many methods for measuring DNA methylation are well known and can be used with the present teaching. All methods require an initial bisulfite modification of the nucleic acid which results in unmethylated cytosine being converted to uracil. This conversion in essence creates a polymorphism which is subsequently measured and representative of the methylation level in the starting DNA. Examples of such methods include bisulfite sequencing, including Pyrosequencing, methylation specific PCR and high resolution melt analysis of PCR amplified bisulfite DNA.

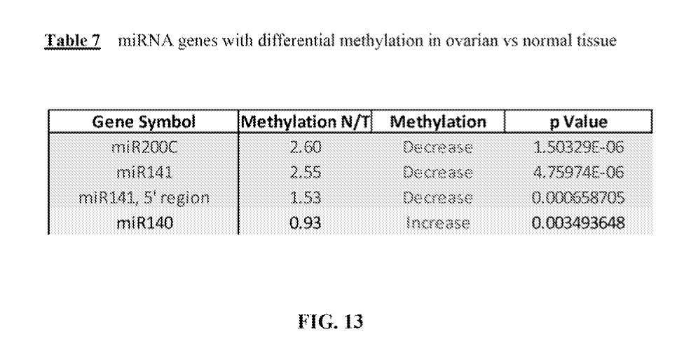

In this example, bisulfite modified DNA from tumor or normal tissue was subjected to methylation analysis using Pyrosequencing. Gene specific primers were used to amplify gene specific regions using polymerase chain reaction (PCR). The amplified products were subjected to quantitiative sequencing using Pyrosequencing technology. The methylation at each CpG site contained within the gene region is quantified as a percentage of the amount of unmethylated cytosine at each site in the starting material. The percentage of methylation at each CpG site in the gene region is averaged together to provide an average methylation level for each gene. The results are shown in Table 7 (FIG. 13).

### Incorporation by Reference

References and citations to other documents, such as patents, patent applications, patent publications, journals, books, papers, web contents, have been made in this disclosure. All such documents are hereby incorporated herein by reference in their entirety for all purposes.

### Equivalents

The representative examples are intended to help illustrate the invention, and are not intended to, nor should they be construed to, limit the scope of the invention. Indeed, various Modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance which can be adapted to the practice of this invention in its various embodiments and equivalents thereof

What is claimed is:

1. A method for determining the presence or risk of ovarian cancer in a human subject, the method comprising:
obtaining a test sample from a human subject;
detecting the test sample obtained from the subject for the presence, amount, or both the presence and amount of one or more genetic biomarkers;
detecting the test sample obtained from the subject for the presence, amount, or both the presence and amount of one or more epigenetic biomarkers; and
transforming the result of genetic biomarker analysis and the result of epigenetic biomarker analysis into one or more parameters useful in determining the presence or risk of ovarian cancer in the subject.

2. The method of claim 1, wherein at least one of the one or more genetic biomarkers provides gene mutation information of the subject.

3. The method of claim 1, wherein at least one of the one or more epigenetic biomarkers provides miRNA expression information of the subject.

4. The method of claim 1, wherein at least one of the one or more epigenetic biomarkers provides aberrant DNA methylation information of the subject.

5. The method of claim 1, wherein the one or more genetic biomarkers are selected from Table 3A and Table 3B and wherein the one or more epigenetic biomarkers are selected from Table 4.

6. The method of claim 1, comprising three or more genetic biomarkers selected from Table 3A and Table 3B and three or more epigenetic biomarkers selected from Table 4.

7. The method of claim 6, comprising five or more genetic biomarkers selected from Table 3A and Table 3B and five or more epigenetic biomarkers selected from Table 4.

8. The method of claim 1, comprising genetic biomarkers are and epigenetic biomarkers are selected from Table 5.

9. The method of claim 1, wherein the one or more genetic biomarkers and one or more epigenetic biomarkers are indicative of a distinctive sub-type of ovarian cancer.

10. A multi-marker panel for determining the presence or risk of ovarian cancer in a human subject, the panel comprising one or more genetic biomarkers and one or more epigenetic biomarkers.

11. The multi-marker panel of claim 10, comprising three or more genetic biomarkers and three or more epigenetic biomarkers.

12. The multi-marker panel of claim 11, comprising five or more genetic biomarkers and five or more epigenetic biomarkers.

13. The multi-marker panel of claim 10, wherein the at least one of the one or more genetic biomarkers provides gene mutation information of the subject.

14. The multi-marker panel of claim 10, wherein the at least one of the one or more epigenetic biomarkers provides miRNA expression information of the subject.

15. The multi-marker panel of claim 10, wherein the at least one of the one or more epigenetic biomarker provides aberrant DNA methylation information of the subject.

16. The multi-marker panel of claim 10, wherein the one or more genetic biomarkers are selected from Table 3A and Table 3B and wherein the one or more epigenetic biomarkers are selected from Table 4.

17. A method for determining whether a tumor sample comprises an ovarian cancer cell, comprising detecting polynucleotide expression levels for one or more of genes selected from Table 5 and determining aberrant methylation levels for one or more of biomarkers from Table 5.

18. The method of claim 17, comprising detecting polynucleotide expression levels for three or more of genes selected from Table 5 and determining aberrant methylation level for three or more of markers from Table 5.

19. The method of claim 17, comprising detecting polynucleotide expression levels for five or more of genes selected from Table 5 and determining aberrant methylation level for five or more of markers from Table 5.

20. A method for determining the presence or risk of cancer in a human subject, the method comprising:
obtaining a test sample from a human subject;
detecting the test sample obtained from the subject for the presence, amount, or both the presence and amount of one or more genetic biomarkers;
detecting the test sample obtained from the subject for the presence, amount, or both the presence and amount of one or more epigenetic biomarkers; and
transforming the result of genetic biomarker analysis and the result of epigenetic biomarker analysis into one or more parameters useful in determining the presence or risk of ovarian cancer in the subject.

21. The method of claim 20, wherein the cancer is selected from the group consisting of: ovarian cancer, cervical cancer, colon cancer and breast cancer.
